(19) United States
(12) Patent Application Publication
(10) Pub. No.: US 2025/0041203 A1
(43) Pub. Date: Feb. 6, 2025

(54) Coconut Oil and Xylitol-Based Mouthwash Composition for Oral Care and Method of Use Thereof

(71) Applicant: SERVIDENCE INC., Saint-Hyacinthe (CA)

(72) Inventor: Jacques Veronneau, Saint-Hyacinthe (CA)

(21) Appl. No.: 18/652,088

(22) Filed: May 1, 2024

Related U.S. Application Data
(60) Provisional application No. 63/499,275, filed on May 1, 2023.

(51) Int. Cl. A61K 8/9794; A61K 8/34; A61Q 11/00

(52) U.S. Cl. CPC A61K 8/9794; A61K 8/345; A61Q 11/00; A61K 2800/92

(57) ABSTRACT

A coconut oil and xylitol-based mouthwash composition for oral care that addresses oral health issues. The mouthwash contains coconut oil and xylitol as active ingredients and has been tested in vivo on dental patients, pregnant women, and orthodontic patients. The formulation is similar to other mouthwashes, consisting of water-glycerine mixture, sweetener, surfactant, preservative, colorant, and flavoring agent. The present invention offers an effective and natural solution for maintaining oral health and addressing various oral and throat-related issues. The natural formulation was found effective against various periodontal and caries bacteria. The coconut oil and xylitol-based mouthwash composition provides an effective and natural solution for maintaining oral hygiene, addressing various oral and throat-related issues.

### CROSS-REFERENCE TO RELATED APPLICATIONS

The present patent application claims the benefits of priority of U.S. Provisional Patent Application No. 63/499,275, entitled “Coconut Oil and Xylitol-Based Mouthwash Composition for Oral Care and Method of Use Thereof”, and filed at the United States Patent and Trademark Office on May 1, 2023, the content of which is incorporated herein by reference.

### FIELD OF THE INVENTION

The present invention generally relates to the field of oral care and oral hygiene products. More precisely, the present invention relates to the field of mouthwash compositions for oral care.

### BACKGROUND OF THE INVENTION

Dental caries and periodontal disease are major contributors to tooth loss, accounting for 95% of missing teeth. While brushing, flossing, and maintaining a healthy diet are essential for oral hygiene, there is a need for additional products to enhance their effectiveness. Mouthwashes are a popular choice, with over 200 products available in the market. These products aim to provide two main oral health benefits: anti-caries and antibacterial effects. Sodium fluoride is the primary active ingredient in mouthwashes designed for anti-caries effect. In contrast, mouthwashes designed for antibacterial effect (which is the target of approximately 50% of all mouthwashes) contain essential oils, mainly menthol, as the main active ingredient.

Current commercial oral rinse products typically use chemotherapeutic agents such as fluoride, essential oils, and chlorhexidine as antimicrobial drugs and anti-caries compounds. However, the use of chlorhexidine in mouthwashes can alter the composition of the oral microbiome and has several undesirable side-effects such as tooth staining, taste alteration, calculus formation, and oral mucosa desquamation/irritation. Moreover, non-compliance with current oral rinse product use recommendations is a significant issue mainly due to their bad, burning or too strong sensation or taste.

Given the increasing public interest in natural or herbal-based healthcare products, there is a growing need for natural mouthwash products that provide effective oral hygiene without the undesirable side-effects of existing products. Therefore, a natural mouthwash composition that is effective, safe, and easy to use could provide an alternative option for consumers looking for a more natural approach to oral hygiene.

### SUMMARY OF THE INVENTION

The shortcomings of the prior art are generally mitigated by the present invention, a coconut oil and xylitol-based mouthwash composition and a method of use thereof.

The present invention is a natural mouthwash which contains as active ingredients coconut oil and xylitol and has been tested to assess its effectiveness on taste perception, antibacterial effects, gingivitis, bleeding issues, throat and oropharynx inflammations in dental patients, pregnant women, and orthodontic patients. Coconut oil contains lauric acid, which has anti-inflammatory and antimicrobial properties, while xylitol is a naturally occurring antibacterial agent.

The other ingredients of the formulation of the present invention are somewhat similar to other main mouthwashes, and are selected from a water-glycerine mixture, sweetener, surfactant, preservative, colorant and flavoring agent.

The present invention offers an effective and natural solution for maintaining oral health and addressing various oral and throat-related issues.

Other and further aspects and advantages of the present invention will be obvious upon an understanding of the illustrative embodiments about to be described or will be indicated in the appended claims, and various advantages not referred to herein will occur to one skilled in the art upon employment of the invention in practice.

### BRIEF DESCRIPTION OF THE DRAWINGS

The above and other aspects, features and advantages of the invention will become more readily apparent from the following description, reference being made to the accompanying drawings in which:

FIG. 1 is a graph showing the antibacterial effect of the present invention on 15 participants in a study.

FIG. 2 is an image representing the various mouthwashes that have been used in a taste study of the present invention.

### DETAILED DESCRIPTION OF THE INVENTION

A novel coconut oil and xylitol-based mouthwash composition for oral care and a method of use thereof will be described hereinafter. Although the invention is described in terms of specific illustrative embodiments, it is to be understood that the embodiments described herein are by way of example only and that the scope of the invention is not intended to be limited thereby.

The invention is a new and improved mouthwash formulation that provides effective oral hygiene by killing harmful bacteria in the mouth and preventing the formation of plaque. This mouthwash is made using a combination of natural ingredients that are safe for human consumption and do not cause any harmful side effects.

According to a preferred embodiment of the present invention, Table 1 below presents a typical formulation of an embodiment of the present invention in which xylitol and coconut oil are the main active ingredients.

TABLE 1
Formulation of an embodiment of the mouthwash composition
Ingredients

| INCI (International Nomenclature | % by weight (plus |
|---|---|
| of Cosmetic Ingredients) names | or minus 0.05%) |
| AQUA | 85.399960 |
| SORBITOL | 6.200000 |
| XYLITOL | 5.000000 |
| PEG-40 HYDROGENATED CASTOR OIL | 2.000000 |
| COCOS NUCIFERA OIL | 0.500000 |
| SODIUM BENZOATE | 0.500000 |
| AROMA | 0.300000 |
| SODIUM SACCHARIN | 0.100000 |
| CI 42090 | 0.000040 |
| TOTAL | 100.000000 |

The mouthwash composition described herein comprises natural ingredients xylitol and coconut oil, which have been found to possess antibacterial properties. While xylitol mouthwash has been shown to be effective against cariogenic bacteria in vitro, coconut oil mouthwash has up to now limited clinical effectiveness and compliance due to the typical requirement for 15 minutes of liquid pulling.

Therefore, the present invention aims to validate the antibacterial effectiveness of the combination of the natural ingredients xylitol and coconut oil on key bacteria involved in caries and periodontal disease processes. The mouthwash is applied twice a day for 30 seconds each time over a three-week period, targeting the following periodontal and caries bacteria: Porphyromonas gingivalis (Pg), Treponoma Denticola (Td), Tannerella forsythia (Tf), Aggregatibacter actinomycetemcomitans (Aa), Fusobacterium nucleatum (Fn), Peptostreptococcus micros (Pm), Candida albicans (Ca), and Mutans streptococci (MS). The critical threshold values for each of the bacteria are provided in Table 2 below.

TABLE 2
Periodontal and caries targeted bacteria

| Abbreviation | HR periodontal bacteria | Critical Threshold |
|---|---|---|
| Pg | Porphyromonas Gingivalis | 5 × (10⁴-10⁵) |
| Td | Treponoma Denticola | 3 × (10²-10³) |
| Tf | Tannerella Forsythia | 5 × (10⁴-10⁵) |
| Aa | Aggregatibacter | 4 × (10³-10⁴) |
| | Actinomycetemcomitans | |
| Abbreviation | MR periodontal bacteria | Critical Threshold |
| Fn | Fusobacterium Nucleatum | 5 × (10⁶-10⁷) |
| Pm | Peptostreptococcus Micros | 5 × (10⁴-10⁵) |
| Ca | Candida Albicans | 3 × (10²-10³) |

| Abbreviation | Caries bacteria | Critical Threshold |
|---|---|---|
| MS | Mutans Streptococci | 5 × (10⁵-10⁶) |

In a study to put the antibacterial effect of the present invention to the test, 15 voluntary participants were recruited to assess the prevalence of Table 2 bacteria before and after rinsing with the embodiment of Table 1 of the present invention. The method for collecting saliva and plaque samples was based on the OraVital® (Ontario) company method, and the samples were analyzed by an external expert from the University of Toronto to avoid internal desirable bias in the results.

The sample analysis included DNA preparation and a microbial analysis down to the species level. The analysis method was led by Dr. Barbour, Ph.D. from the Oral Microbiome Biofilms and Probiotics attached to the Faculty of Dentistry at the University of Toronto. A serial dilution was done with each sample, and colony-forming units (c.f.u.) were calculated before and after treatment for each participant. Quantitative PCR was performed to estimate the bacterial copy numbers of eight bacterial pathogens, including P. gingivalis, T. denticola, T. forsythia, P. micra, C. albicans, A. actinomycetemcomitans, F. nucleatum, and S. mutans.

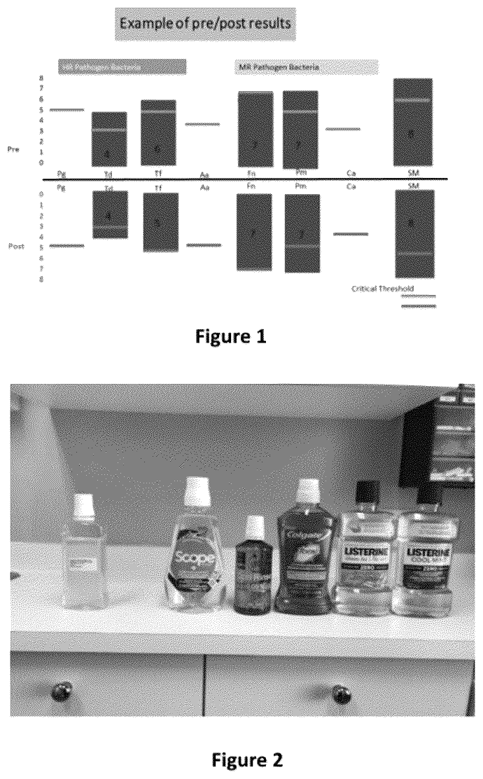

Referring to FIG. 1, after sample collection and analysis of each participant, a preferred embodiment of the invention (see Table 1) was found to decrease the bacteria at the threshold level or lower by an average of 45 times, with a total of 27 significant decreasing targeted high periodontal risk bacteria. The results in FIG. 1 show a clear reduction in global bacteria prevalence, with a total of 46 logs bacteria or a billion bacterial reduction within three weeks. The presence of xylitol at an approximately 5% (by weight) dosage, and the method of rinsing twice a day for 30 seconds were considered to be the key factors in achieving these results.

Then, to test the efficacy of a preferred embodiment of the present invention (see Table 1) in resolving and preventing inflammation, a study was conducted with 15 participants suffering from various oral, throat, and pharynx discomforts. These participants had consulted with various medical experts and had tried different medications, but had not seen improvements in their quality of life.

After using the present invention for three weeks, twice a day, for 30 seconds, participants reported positive results. They unanimously agreed that the rinse had a positive impact on their oral health and wished that it was available on the market. Participants with dry, wet, or hacking coughs during the day reported a significant reduction or complete cessation of their coughing. One participant who experienced a dry throat feeling at night reported better sleep after using the present mouthwash. Gingival bleeding was also significantly reduced after just one week of use for the majority of participants.

Participants reported feeling a soft, oily sensation in their teeth and gingival tissue after using the formulation of the present invention, indicating that it had a positive effect on their oral tissues. Those who had experienced tingling sensations in the back of their throat due to allergies reported that this sensation disappeared after two weeks of use. Participants who felt secretions in the back of their throat found that the rinse helped to facilitate the removal of these secretions. Those with autoimmune diseases and lacy-white lesions, ulcers, or canker or reddish sores in their mouth felt relieved and secure after using the mouthwash.

In terms of subjective quantitative results, some participants were able to assess their improvement before and after using the present invention. One participant reported a 100% reduction in bleeding after three weeks of use, while the extent of tissue inflammation was reduced by 40% in the same period. Participants with secretions caught in the back of their throat were able to expectorate them and clean 50% of their secretions. Participants with different types of coughing saw improvements, reducing their coughing by 60%. Overall, participants reported that the present invention had a positive impact on their quality of life and expressed frustration that medical professionals were not aware of this rinse.

Now referring to FIG. 2, to evaluate the new taste of the present invention, a study was conducted with 50 participants accepting to be exposed daily (twice a day) to the mouthwash for a total period of three weeks. Taste is a homeostatic function that conveys valuable information, such as energy density, readiness to eat, or toxicity of foodstuffs. Taste is not limited to the oral cavity but affects multiple physiological systems. Before the study started, each participant signed an individual consent form, and a self-reported questionnaire was given to all participants to fill out at the end of their experience. The questionnaire collected basic demographic information, specific oral health problem pre-conditions, other rinse experiences, medical treatments applied for the condition, taste and feeling around the mouthwash, and the effect of the daily 30 seconds rinsing experience on their condition. The data collection was anonymous, kept confidential, and stored securely.

Each participant was asked to visit the dental clinic three weeks later and allowed a video capture.

After rinsing with the mouthwash for 30 seconds, all participants found it to have a better taste than existing mouthwashes on the market as shown in FIG. 2. Participants identified Listerine® as the most prevalent tested brand. They generally found that existing mouthwashes were too strong, abrasive, or corrosive, and they felt like their mucous membranes were superficially anesthetized, their mouth tissues were burning, and their teeth were melting.

Although a preferred embodiment of the invention contains 5% (plus or minus 0.05%) of xylitol and 0.5% (plus or minus 0.05%) coconut oil, the concentration of these ingredients can vary as follows: 5% to 15% (plus or minus 0.05%) of xylitol and 0.2% to 3% (plus or minus 0.05%) of coconut oil.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

1. A mouthwash composition comprising, as active ingredients, coconut oil and xylitol.

2. A mouthwash composition as claimed in claim 1, wherein the coconut oil comprises between 0.2% and 3% of the mouthwash composition.

3. A mouthwash composition as claimed in claim 1, wherein the xylitol comprises between 5% and 15% of the mouthwash composition.

4. A mouthwash composition as claimed in claim 1, wherein the coconut oil comprises between 0.2% and 3% of the mouthwash composition the xylitol comprises between 5% and 15% of the mouthwash composition.

5. A mouthwash composition as claimed in claim 4, wherein the coconut oil comprises 0.5% (plus or minus 0.05%) of the mouthwash composition the xylitol comprises 5% (plus or minus 0.05%) of the mouthwash composition.

6. A method of use of the mouthwash composition of claim 1 substantially in accordance with the above description.
